(19) United States
(12) Patent Application Publication
(10) Pub. No.: US 2024/0173565 A1
(43) Pub. Date: May 30, 2024

(54) PHOTOTHERAPY LASER LIGHT APPARATUS

(71) Applicant: Arthur Flick, Lakemont, GA (US)

(72) Inventor: Arthur Flick, Lakemont, GA (US)

(21) Appl. No.: 18/496,370

(22) Filed: Oct. 27, 2023

Related U.S. Application Data
(60) Provisional application No. 63/385,016, filed on Nov. 30, 2022.

(51) Int. Cl. A61N 5/067; A61N 2/02; A61N 5/06

(52) U.S. Cl. CPC A61N 5/067; A61N 2/02; A61N 5/0613; A61N 2005/0632; A61N 2005/0645; A61N 2005/0659; A61N 2005/0663

(57) ABSTRACT

A light emitting apparatus including a base and a plurality of light sources is disclosed. The light sources may be disposed on the base and configured to emit coherent light. The plurality of light sources comprises a first set of light sources and a second set of light sources. The first set of light sources may be configured to emit pulses of coherent light modulated at a first frequency from a plurality of frequencies based on command signals obtained from a frequency generator. The second set of light sources may be configured to emit pulses of coherent light modulated at a second frequency, from the plurality of frequencies, based on the command signals obtained from the frequency generator. The first frequency may be different from the second frequency.

### CROSS-REFERENCE TO RELATED APPLICATION

This application claims priority to and the benefit of U.S. provisional application No. 63/385,016, filed Nov. 30, 2022, which is hereby incorporated by reference herein in its entirety.

### TECHNICAL FIELD

The present disclosure relates to a phototherapy laser light apparatus and more particularly to a phototherapy laser light apparatus including a plurality of laser light sources modulated at a plurality of low frequencies that may be configured to emit coherent light directly into a human/animal body or via a solvent and/or a solute.

### BACKGROUND

A majority of people today have long working hours and lack active lifestyle. Such hectic lifestyle results in many health-related issues. Health related issues further get accentuated in old age. For example, people at old age are known to suffer from knee pain, dental pain or swelling, neck pain, back pain, slow cognitive impairment, diabetes, seizures, and/or the like. In modern times, even people at young age suffer from one or more of these ailments.

Many people consume medications to treat their ailments. Medications are known to cause side effects. Along with or alternative to medications, some modern medical practitioners treat their patients using phototherapy, magneto therapy, and/or the like. Such treatment methods are subject of ongoing research and clinical trials. While efficacy of such treatment methods are now well known, improvements are required to further enhance their efficacy.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1 depicts an example light emitting apparatus in accordance with the present disclosure.

FIG. 2 depicts an enlarged view of the light emitting apparatus of FIG. 1 in accordance with the present disclosure.

FIG. 3 depicts two light emitting apparatuses attached to a frequency generator in accordance with the present disclosure.

FIG. 4 depicts a plurality of light emitting apparatuses attached to two expanders operated by a frequency generator in accordance with the present disclosure.

FIG. 5 depicts an example magnetic field generating apparatus in accordance with the present disclosure.

FIG. 6 depicts a block diagram of an example frequency generator in accordance with the present disclosure.

FIG. 7 depicts a block diagram of an example computer application associated with a frequency generator in accordance with the present disclosure.

FIG. 8 depicts a first example usage of one or more light emitting apparatuses in accordance with the present disclosure.

FIG. 9 depicts a second example usage of one or more light emitting apparatuses in accordance with the present disclosure.

FIG. 10 depicts a third example usage of one or more light emitting apparatuses in accordance with the present disclosure.

FIG. 11 depicts a fourth example usage of a light emitting apparatus in accordance with the present disclosure.

FIG. 12 depicts a fifth example usage of one or more light emitting apparatuses in accordance with the present disclosure.

FIG. 13 depicts a sixth example usage of one or more light emitting apparatuses and magnetic field generating apparatuses in accordance with the present disclosure.

FIG. 14 depicts an example light emitting apparatus integrated with a fluid vessel in accordance with the present disclosure.

FIG. 15 depicts one or more example light emitting apparatuses connected with an intravenous solution bag in accordance with the present disclosure.

FIGS. 16A, 16B, 16C depict example tables illustrating a plurality of low frequencies associated with pulses of coherent light in accordance with the present disclosure.

### DETAILED DESCRIPTION

### Overview

The present disclosure describes a phototherapy light emitting apparatus (“apparatus”) that may be used to perform bio-stimulating phototherapy on a human or an animal body. The apparatus may include a plurality of light sources that may be configured to emit coherent light. In some aspects, the light sources may be low-level laser light sources that may be configured to emit laser light beams, which may be configured to penetrate into human/animal skin to cause phototherapy. The light sources may emit pulses of coherent light modulated at a plurality of low frequencies (ranging from 0.1 Hz to 5,000 Hz) based on command signals obtained from a frequency generator. The frequency generator may be configured to power the light sources as well as provide the low frequencies at which the light sources may be modulated.

In an exemplary aspect, the apparatus may include seven sets of light sources, and each set of light sources may include six light sources. The light sources in each set may be disposed in a hexagonal pattern in the apparatus. The frequency generator may modulate each set of light sources at a different frequency simultaneously. For example, a first set of light sources may be modulated at a first frequency, a second set of light sources may be modulated at a second frequency, a third set of light sources may be modulated at a third frequency, etc. During operation, the apparatus may be placed in proximity to a human/animal skin such that the light sources may face the human/animal skin. Upon activation and modulation of each set of light sources at a different frequency, the light sources may begin to emit pulses of coherent light (at respectively frequencies), which may penetrate the human/animal skin to cause phototherapy.

In some aspects, the apparatus may operate in conjunction with a magnetic field generating apparatus, which may be placed in proximity to the human/animal skin along with the apparatus described above. In an exemplary aspect, the magnetic field generating apparatus may include seven magnetic coils that may be disposed in the magnetic field generating apparatus in the same hexagonal pattern as the seven sets of light sources may be disposed in the apparatus. The magnetic field generating apparatus may also be operated by the frequency generator. Specifically, based on the command signals obtained from the frequency generator, the seven magnetic coils included in the magnetic field generating apparatus may generate pulses of magnetic field that may be modulated in the same seven frequencies as the seven sets of light sources in the apparatus. The pulses of magnetic field generated by the seven magnetic coils may enter the human/animal skin when the magnetic field generating apparatus may be placed in proximity to the human/animal skin, causing magneto therapy. In some aspects, the pulses of magnetic field generated by the seven magnetic coils may be synchronized with the pulses of coherent light beams emitted from the seven sets of light sources included in the apparatus, for enhanced efficacy of treatment in a patient.

In further aspects, the apparatus may be integrated with or attached with a fluid vessel that may be configured to store a solvent with or without a solute. The pulses of coherent light beam emitted from the apparatus may interact with the solvent and/or the solute stored in the fluid vessel, and then made to penetrate into the human/animal skin, for enhanced phototherapy.

In an exemplary aspect, the solvent may be water, although other polar and non-polar solvents may also be used. The solute may be, for example, one or more of anticoagulated whole blood and blood plasma, biological cells and tissues, platelet rich plasma, homeopathic preparations, herbal preparations, crystals, minerals and elements, vitamins, flowers, industrial chemicals, colors, herbal and plant remedies, nutraceuticals, pharmaceuticals, biomolecular substances, flower essences, essential oils, gem stones, and/or the like.

The present disclosure discloses a system including a phototherapy light emitting apparatus, a magnetic field generating apparatus and a fluid vessel that may be used to perform enhanced phototherapy and magneto therapy on a patient. The pulses of coherent light beams emitted at the plurality of low frequencies from the phototherapy light emitting apparatus results in enhanced phototherapy. Further, when the pulses of coherent light beams are made to penetrate into the human/animal skin along with the synchronized or non-synchronized pulses of magnetic field at the same plurality of low frequencies generated from the magnetic field generating apparatus, the patients may experience simultaneous phototherapy and magneto therapy, thereby enabling quick patient recovery.

These and other advantages of the present disclosure are provided in detail herein.

### Illustrative Embodiments

The disclosure will be described more fully hereinafter with reference to the accompanying drawings, in which example embodiments of the disclosure are shown, and not intended to be limiting.

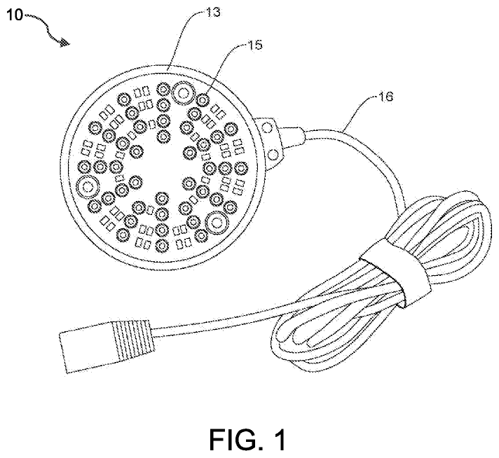

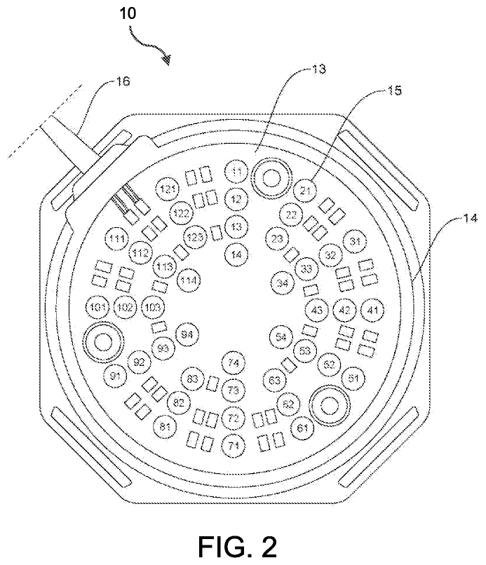

FIG. 1 depicts an example light emitting apparatus 10 (apparatus 10) in accordance with the present disclosure. While describing FIG. 1, references will be made to FIG. 2, which depicts an enlarged view of the apparatus 10.

The apparatus 10 may be a phototherapy laser light emitting apparatus that may be attached to or disposed in proximity of a human body or an animal body to perform bio-stimulating phototherapy. The apparatus 10 may be configured to emit coherent light beam that may penetrate human or animal skin to produce photo-biological reaction, which may treat a plurality of ailments including, but not limited to, knee pain, dental pain or swelling, neck pain, back pain, slow cognitive impairment, diabetes, seizures, and/or the like.

The apparatus 10 may include a base 13, a plurality of light sources 15, a multi-stranded cable 16, and a flexible attachment structure 14 (shown in FIG. 2). In some aspects, the base 13 may be shaped as a circular disc and may be made of material such as plastic, rubber, silicon, metal, and/or the like. In other aspects (not shown), the base 13 may be of any other shape, e.g., elliptical, square, rectangular, hexagonal, pentagonal, etc., without departing from the present disclosure scope.

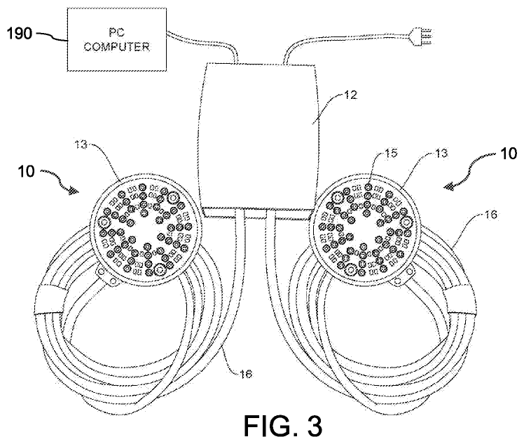

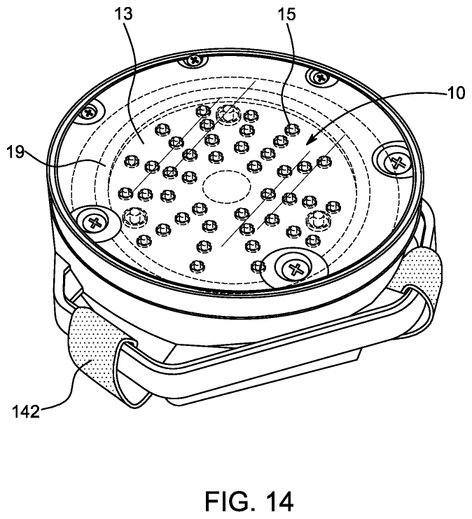

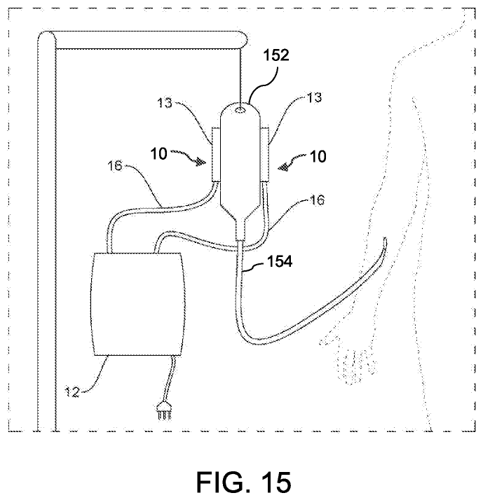

The light sources 15 may be disposed on or housed in the base 13. Each light source 15 may be a low-level laser light source that may be configured to emit coherent light. Further, each light source 15 may be configured to emit pulses of coherent light modulated at a plurality of low frequencies, which may be in a range of 0.1 Hz to 5,000 Hz. The cable 16 may be configured to transfer energy/power and/or command signals from a frequency generator 12 (described below in conjunction with FIG. 3) to operate the light sources 15 modulated at the plurality of low frequencies. The coherent light emitted from each light source 15 may be configured to penetrate into the human or animal skin to produce photo-biological reaction, as described above. The flexible attachment structure 14 may be made of flexible material such as plastic, rubber, etc., and may enable external fastening means such as straps to be attached to the apparatus 10. The external fastening means may enable an apparatus user to attach the apparatus 10 to a plurality of animal or human body parts, e.g., knee, ankle, wrist, arm, elbow, shoulder, spine, head, etc. that requires phototherapy using the coherent light emitted from the light sources 15. The flexible attachment structure 14 may further enable the user to attach or integrate the apparatus 10 with a fluid vessel 152 (described below in conjunction with FIG. 15) and/or an intravenous tube that may be configured to store intravenous fluids that may be injected to human/animal body for treatment. Example usage of the apparatus 10 with the fluid vessel is described later below in conjunction with FIGS. 14 and 15.

A person ordinarily skilled in the art may appreciate that a laser light source (e.g., each light source 15) may generate and emit intense, monochromatic, coherent, and highly collimated beam of light. To produce a required photo-biological reaction to treat a particular user ailment without adversely affecting the user's skin, the laser light emitted from each light source 15 may be required to pass through the skin without causing injury to skin's dermal structures. Further, the laser light may be required to induce photon absorption by endogenous photon-acceptor molecules in the skin. The energy absorbed by the molecules may cause chemical reactions that may treat the user's ailment, without altering temperature of surrounding tissues or causing any adverse effect.

Further, a person ordinarily skilled in the art may appreciate that wavelength, intensity, power density/irradiance, and/or the like of laser radiation/light emitted by the light sources 15 have direct correlation with the depth of tissue penetration in the user's skin, and the effectiveness and photo-biological impacts of the laser radiation/light to treat user's ailment. In accordance with the present disclosure, the light sources 15 may be low-level laser light sources that deliver laser light of a few milliwatts/cm² power. In an exemplary aspect, each light source 15 may have an associated power rating of less than 1 Watt. In a preferred aspect, each light source 15 may have an associated power rating of less than 100 milliwatts, and more particularly less than 5 milliwatts. Further, each light source 15 may emit a coherent light beam that may penetrate five to six centimeters inside an animal skin or a human skin.

In further aspects, each light source 15 may be configured to emit coherent light having a wavelength in a range of 390 to 1,600 nanometer (nm). In a preferred aspect, each light source 15 may emit coherent light with a wavelength in a range of 600 nm to 1300 nm, more particularly in a range of 600 nm to 900 nm, and more particularly in a range of 640 nm to 680 nm. Further, each light source 15 may emit coherent light having a parallel divergence of 8 to 12 degrees and a perpendicular divergence of 27 to 32 degrees. In some aspects, the nature of radiation/light emitted by the light sources 15 is selected to ensure optimal penetration of light into the biological tissue and treatment of user's ailment.

The plurality of light sources 15 may emit coherent light at a single frequency that may be based on the wavelength described above and the medium of light propagation. Further, each light source 15 may emit pulses of coherent light modulated at a plurality of low frequencies, ranging from 0.1 Hz to 5,000 Hz. Specifically, in an exemplary aspect, the plurality of light source 15 may include a predefined count of sets of light sources (e.g., seven sets of light sources), and each set of light sources may be modulated at a different frequency, from the plurality of low frequencies.

In the exemplary aspect depicted in FIG. 2, the apparatus 10/the plurality of light sources 15 may include seven sets of light sources, e.g., a first set of light sources numbered 11, 31, 51, 71, 91 and 111, a second set of light sources numbered 21, 41, 61, 81, 101 and 121, a third set of light sources numbered 12, 32, 52, 72, 92 and 112, a fourth set of light sources numbered 22, 42, 62, 82, 102 and 122, a fifth set of light sources numbered 13, 33, 53, 73, 93 and 113, a sixth set of light sources numbered 23, 43, 63, 83, 103 and 123, and a seventh of light sources numbered 14, 34, 54, 74, 94 and 114. Each set of light sources may include six light sources, and the six light sources in each set of light sources may be disposed/housed in a hexagonal pattern on the base 13. A person ordinarily skilled in the art may appreciate that since many of the naturally occurring structural elements in nature are hexagonal in shape (e.g., structure of a snowflake, structure in which water crystalizes, etc.), placing the light sources 15 in a hexagonal pattern mimics nature and hence enhances efficacy of phototherapy caused by the coherent light beams emitted from the light sources 15.

As shown in FIG. 2, the 42 light sources 15 included in the seven sets of light sources may be arranged in four concentric circles with diminishing radii. For example, the first and second sets of light sources may be housed in the base 13 at an outermost circle with a maximum radius, the third and fourth sets of light sources may be housed in the base 13 at a first middle circle with a first radius which may be less than the maximum radius, the fifth and sixth sets of light sources may be housed in the base 13 at a second middle circle with a second radius which may be less than the first radius, and the seventh set of light sources may be housed in the base 13 at an innermost circle with a minimum radius. The outermost circle, the first middle circle and the second middle circle may include 12 light sources each, spaced 30 degrees apart relative to a base center point. Further, the innermost circle may include six light sources spaced 60 degrees apart relative to the base center point.

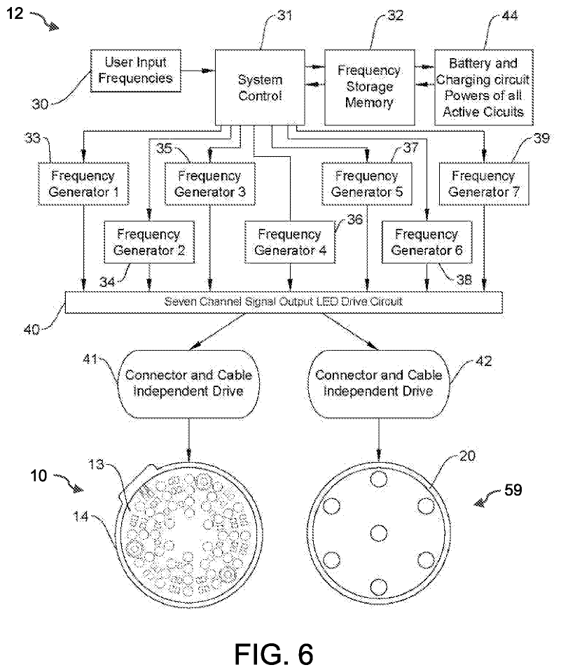

As described above, the light sources 15 may be modulated at the plurality of low frequencies. Specifically, each light source 15 may emit pulses of coherent light modulated at any one frequency of the plurality of low frequencies. In some aspects, each light source from the 42 light sources 15 may be modulated at one specific frequency based on command signals obtained from the frequency generator 12 (described below in conjunction with FIG. 6). Further, the light sources included in each set of light sources may be modulated at the same frequency. For example, the first set of light sources may emit pulses of coherent light modulated at a first frequency (from the plurality of low frequencies) based on the command signals obtained from the frequency generator 12. Similarly, the second set of light sources may emit pulses of coherent light modulated at a second frequency (from the plurality of low frequencies), the third set of light sources may emit pulses of coherent light modulated at a third frequency (from the plurality of low frequencies), the fourth set of light sources may emit pulses of coherent light modulated at a fourth frequency (from the plurality of low frequencies), the fifth set of light sources may emit pulses of coherent light modulated at a fifth frequency (from the plurality of low frequencies), the sixth set of light sources may emit pulses of coherent light modulated at a sixth frequency (from the plurality of low frequencies), and the seventh set of light sources may emit pulses of coherent light modulated at a seventh frequency (from the plurality of low frequencies). In some aspects, the first frequency, the second frequency, the third frequency, the fourth frequency, the fifth frequency, the sixth frequency and the seventh frequency may be different from each other. In other aspects, one or more frequencies from the first-seventh frequencies may be same or different from each other.

The plurality of light sources 15 may be modulated at one of the first-seventh frequencies simultaneously, and may simultaneously emit pulses of coherent light beams into the human/animal skin based on the frequencies at which the light sources 15 may be modulated. Simultaneous penetration of coherent light modulated at different frequencies into the human/animal skin results in enhanced therapeutic outcome as compared to conventional phototherapy apparatuses. In a preferred aspect, each of the first to seventh frequencies is less than 5,000 Hz, which results in enhanced therapeutic outcome.

In some aspects, the first to seventh frequencies may themselves be modulated at an eighth frequency, which may be less than the lowest of the first to seventh frequencies. In an exemplary aspect, the eighth frequency may be less than 16 Hz. In some aspects, the first to seventh frequencies may be modulated at the eighth frequency when the coherent light emitted from the plurality of light sources 15 may pass through a solvent and/or a solute, and then targeted towards a human or an animal body part. Examples of solvent and solute are described later below in conjunction with FIGS. 14 and 15.

In some aspects, a top portion of the base 13 (or base top portion) may include a transparent material (not shown) that may enable the coherent light emitted from the plurality of light sources 15 to un-obstructively pass the apparatus 10 and penetrate into the human/animal skin. In other aspects, the top portion of the base 13 may include a transparent material that may enable the coherent light emitted from the plurality of light sources 15 to be partially obstructed by a predefined geometric pattern that may be overlaid on the surface of the top portion. A base bottom portion (in which the plurality of light sources 15 may be housed) may include an opaque material that may inhibit/block the coherent light emitted from the plurality of light sources 15 to pass through the base bottom portion, thereby preventing loss of light.

FIG. 3 depicts two light emitting apparatuses 10 attached to the frequency generator 12 in accordance with the present disclosure. The apparatuses 10 may be electrically and communicatively coupled with the frequency generator 12 via the cables 16. The frequency generator 12 may be configured to provide power/energy to the plurality of light sources 15 to enable light source operation. The frequency generator 12 may be further configured to transmit command signals to the seven sets of light sources described above, to modulate each set of light sources at one of the first-seventh (low) frequencies. The frequency generator 12 may be further configured to transmit a command signal (e.g., an “eighth” command signal) to all seven sets of light sources described above to modulate all seven sets of light sources simultaneously with an eighth frequency less than the lowest frequency within all seven sets (as described above).

In some aspects, the frequency generator 12 may include an in-built battery (not shown) that may power frequency generator operation. In additional aspects, the frequency generator 12 may be powered by a utility power source (e.g., via a power socket mounted on a wall of a house/building). In further aspects, the user may control frequency generator operation (e.g., switch ON the light sources 15, change and/or set the first-seventh frequencies, the eighth frequency, etc.) by using a computer 190 that may be communicatively coupled with the frequency generator 12 via a wired connection or a wireless communication network. In some aspects, the computer 190 may execute an application/software that may enable the user to conveniently control the frequency generator operation.

In the exemplary aspect depicted in FIG. 3, the two apparatuses 10 may be attached to different user's body parts, and the frequency generator 12 may control operation of both the apparatuses 10 simultaneously. In some aspects, the frequency generator 12 may modulate the two apparatuses 10 with same sets of low frequencies, e.g., first-seventh frequencies.

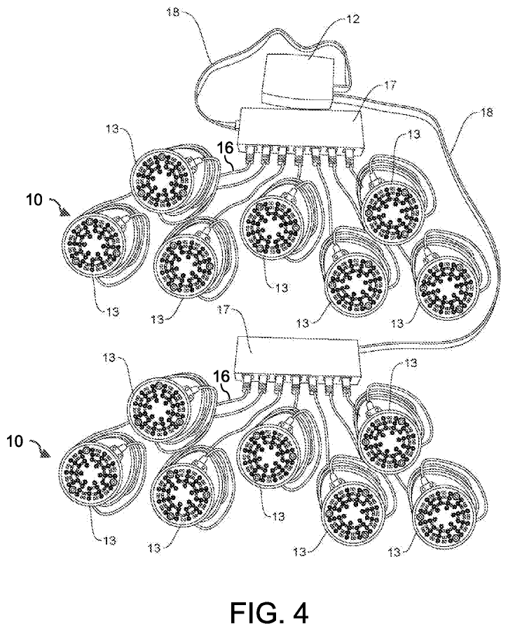

FIG. 4 depicts a plurality of light emitting apparatuses 10 attached to two expanders 17 operated by the frequency generator 12 in accordance with the present disclosure. Each expander 17 may be electrically and communicatively coupled with the frequency generator 12 via expander cables 18. Further, each apparatus 10 may be electrically and communicatively coupled with the expander 17 via the cable 16. In the exemplary aspect depicted in FIG. 4, each expander 17 is connected with seven apparatuses 10. The present disclosure is not limited to such an arrangement. In other aspects, each expander 17 may be connected with more or less than seven apparatuses 10.

In some aspects, each expander 17 may be powered independently via the utility power source or via an in-built expander battery (not shown). Further, all 14 apparatuses 10 connected to the frequency generator 12 via the expanders 17 may be synchronized to radiate the same seven frequencies (i.e., the first-seventh frequencies) at the same time duration and in the same geometric pattern as described above in conjunction with FIGS. 1 and 2. In the exemplary aspect depicted in FIG. 4, the frequency generator 12 may transmit command signals to each expander 17 via the expander cables 18, and the expanders 17 may then in turn cause light source operation and modulate each light source with respective low frequency based on the command signals obtained from the frequency generator 12.

In some aspects, each expander 17 may be connected with a plurality of apparatuses 10, as shown in FIG. 4. In other aspects (not shown), one or more expanders may be further connected with other expanders (which may be connected with the plurality of apparatuses 10) to form a daisy chain series.

In the exemplary aspect depicted in FIG. 4, the plurality of apparatuses 10 may be attached to a plurality of human/animal body parts simultaneously and operated by the frequency generator 12 concurrently, to enhance the effect of bio-stimulating phototherapy in the human/animal.

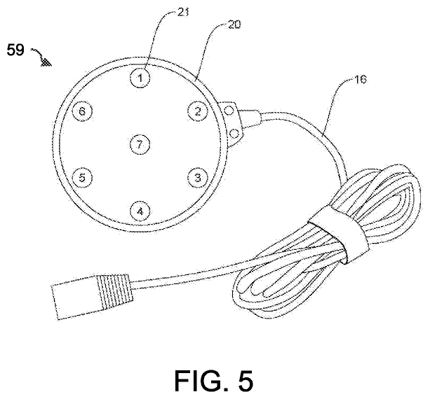

FIG. 5 depicts an example magnetic field generating apparatus 59 (apparatus 59) in accordance with the present disclosure. The apparatus 59 may be configured to generate and emit magnetic field that may penetrate into human/animal skin and be used for magneto therapy. Specifically, the user may attach or place the apparatus 59 in proximity to a human/animal body part (e.g., knee, ankle, leg, spine, etc.) and cause the apparatus 59 to emit pulses of magnetic field that may penetrate human/animal skin and be used to perform magneto therapy.

The apparatus 59 may include a base 20 (which may be similar to the base 13), a plurality of magnetic inductor coils 21 (or magnetic coils 21) and the cable 16. Although the base 20 is shown as being circular in shape in FIG. 5, the present disclosure is not limited to such a base shape. In other aspects, the base 20 may be elliptical, square, rectangular, hexagonal, pentagonal, etc. in shape, without departing from the present disclosure scope. In some aspects, the frequency generator 12 may control magnetic field generating apparatus' operation via the cable 16 in a similar manner as the frequency generator 12 controls the operation of the apparatus 10.

In the exemplary aspect depicted in FIG. 5, the apparatus 59 is shown to include seven magnetic coils 21, i.e., a first magnetic coil 1, a second magnetic coil 2, a third magnetic coil 3, a fourth magnetic coil 4, a fifth magnetic coil 5, a sixth magnetic coil 6 and a seventh magnetic coil 7. In some aspects, the seven magnetic coils 21 may generate and emit seven frequency specific magnetic fields that may penetrate the human/animal skin to cause magneto therapy. The seven magnetic coils 21 may be disposed or housed in the base 20 in a hexagonal pattern, with the first-sixth magnetic coils 1-6 housed in a hexagon (60 degrees apart relative to an apparatus center point) around the seventh magnetic coil 7 that may be disposed/housed at the apparatus center point.

In some aspects, each magnetic coil from the first-seventh magnetic coils 1-7 may be configured to generate and emit pulses of magnetic field modulated at one of the first-seventh frequencies described above, based on command signals obtained from the frequency generator 12. Specifically, the pulses of magnetic field generated by each magnetic coil may be synchronized with the pulses of coherent light emitted by the light sources 15, when the apparatus 10 and the apparatus 59 together may be connected with the frequency generator 12. For example, the first magnetic coil 1 may be configured to generate and emit pulses of magnetic field at the first frequency based on the command signals obtained from the frequency generator 12, and the pulses of magnetic field generated by the first magnetic coil 1 may be synchronized with the pulses of coherent light emitted by the first set of light sources described above. Similarly, the second magnetic coil 2 may be configured to generate and emit pulses of magnetic field at the second frequency based on the command signals obtained from the frequency generator 12, and the pulses of magnetic field generated by the second magnetic coil 2 may be synchronized with the pulses of coherent light emitted by the second set of light sources described above. In a similar manner, the third magnetic coil 3, the fourth magnetic coil 4, the fifth magnetic coil 5, the sixth magnetic coil 6 and the seventh magnetic coil 7 may be configured to generate and emit pulses of magnetic field at the third, fourth, fifth, sixth and seventh frequencies respectively, based on the command signals obtained from the frequency generator 12. Further, the pulses of magnetic fields generated by respective magnetic coils may be synchronized with the pulses of coherent light emitted by the corresponding sets of light sources.

The user may control operation of the apparatus 59 connected to frequency generator 12 described above via the computer 190. For example, the user may switch ON or OFF and/or modify the first-seventh frequencies by providing user inputs to the computer 190, which in turn may control frequency generator operation (and hence the apparatus operation) based on the user inputs.

FIG. 6 depicts a block diagram of the frequency generator 12 in accordance with the present disclosure. The frequency generator 12 may include or receive user input frequencies 30 (e.g., the first-seventh frequencies described above) from the user via, e.g., a Universal Serial Bus (USB) device. In some aspects, a system controller 31 (or system control 31) may obtain the user input frequencies 30 and store the user input frequencies 30 in a frequency storage memory 32 (or memory 32).

The memory 32 may store programs in code and/or store data for performing various frequency generator operations in accordance with the present disclosure. Specifically, the system control 31 may be configured and/or programmed to execute computer-executable instructions stored in the memory 32 for performing various frequency generator functions in accordance with the disclosure. Consequently, the memory 32 may be used for storing code and/or data code and/or data for performing frequency generator operations in accordance with the present disclosure.

In one or more aspects, the system control 31 may be disposed in communication with one or more memory devices (e.g., the memory 32 and/or one or more external databases (not shown in FIG. 6)). The memory 32 may include any one or a combination of volatile memory elements (e.g., dynamic random-access memory (DRAM), synchronous dynamic random access memory (SDRAM), etc.) and can include any one or more nonvolatile memory elements (e.g., erasable programmable read-only memory (EPROM), flash memory, electronically erasable programmable read-only memory (EEPROM), programmable read-only memory (PROM), etc.).

The memory 32 may be one example of a non-transitory computer-readable medium and may be used to store programs in code and/or to store data for performing various operations in accordance with the disclosure. The instructions in the memory 32 may include one or more separate programs, each of which may include an ordered listing of computer-executable instructions for implementing logical functions.

In some aspects, responsive to obtaining the user input frequencies 30, the system control 31 may transmit command signals to a first frequency generator chip 33, a second frequency generator chip 34, a third frequency generator chip 35, a fourth frequency generator chip 36, a fifth frequency generator chip 37, a sixth frequency generator chip 38 and a seventh first frequency generator chip 39 to respectively generate the first-seventh frequencies described above. Responsive to obtaining the command signals from the system control 31, the first-seventh frequency generator chips 33-39 may synthetize seven independent output frequencies (i.e., the first-seventh frequencies) and transmit the seven frequencies (and/or information associated with the seven frequencies) to a seven channel signal output light emitting diode (LED) drive circuit 40 (or circuit 40) that may be communicatively coupled with each of the first-seventh frequency generator chips 33-39 and the apparatuses 10 and 59. The circuit 40 may transmit the seven frequencies (and/or information associated with the seven frequencies) to the apparatus 10 and 59, to cause respective apparatus operation as described above. In some aspects, the circuit 40 may transmit the seven frequencies (and/or information associated with the seven frequencies) to the apparatus 10 and 59 via connector and cable independent drives 41, 42, as shown in FIG. 6. The apparatus 10 and/or the apparatus 59 may each have a cable (e.g., the cable 16) that plugs into the connector and cable independent drives 41, 42 using individual signal wires. Each signal wire may pass through a wave shaping network and independently illuminate the light sources 15 and/or actuate the magnetic coils 21 based on the frequencies and/or command signals obtained from the system control 31.

In some aspects, the system control 31 and the first-seventh frequency generator chips 33-39 may utilize a precision crystal oscillator time base that may be generated in the system control 31 using Crystal X2 and buffered to ensure accuracy in the frequency generation and timing. The frequency generator 12 may further include a battery and charging circuitry 44 (or battery and charging circuit powers of all active circuits) that may include battery/charge status indicators, voltage regulator, audible operator feedback, and On-Off controls associated with the frequency generator 12.

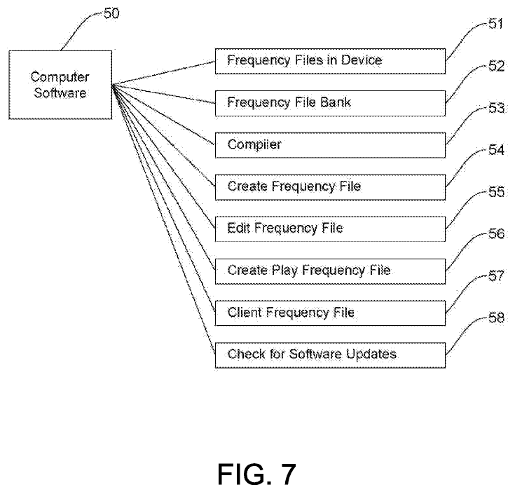

FIG. 7 depicts a block diagram of an example computer application/software 50 associated with the frequency generator 12 in accordance with the present disclosure. Specifically, the computer application 50 may be configured to “program” the frequency generator 12, so that the frequency generator 12 may efficiently control operation of the light sources 15 and the magnetic coils 21.

The computer application 50 may provide a plurality of functions/features to the user. For example, the user may access frequency files in device 51 to view a frequency file and a frequency composition of each frequency file loaded on the frequency generator 12, frequency generator's serial number and a means to start and stop the radiation associated with the light sources 15 and/or the magnetic coils 21. The user may access a frequency file bank 52 to view the categories, the frequency files within each category, the individual frequencies composing each frequency file and an analysis of a count of time durations each frequency may be radiated from the light sources 15. Compiler 53 may create frequency programs composed for a plurality of frequency files. Create frequency file 54 may create frequency files, and edit frequency file 55 may change, add to or subtract frequencies from a frequency file. Create play frequency file 56 may create the seven frequencies described above for a specified time duration/period and cause radiation of the seven frequencies for the specified time duration/period from the apparatus 10 and the apparatus 59. Client frequency files 57 may save frequency programs written/programmed for the user by date and the frequency generator's serial number. Check for software updates 58 may download the most recent software as well as new frequency files that have been clinically tested. In some aspects, the seven frequencies described above may be selected based on the ailment type that needs to be treated in the user.

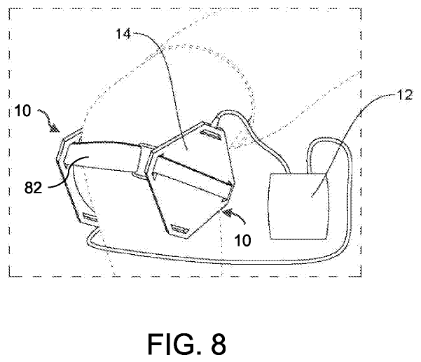

FIG. 8 depicts a first example usage of one or more light emitting apparatuses 10 in accordance with the present disclosure. In the exemplary aspect depicted in FIG. 8, the apparatuses 10 may be used for treating knee pain. The apparatuses 10 may be held in place at the user's knee, as shown in FIG. 8, by a strap 82 or an orthopedic appliance. The apparatus 10 may emit pulses of coherent light beams modulated at the plurality of low frequencies, which may penetrate into the user's skin to treat the knee pain.

In some aspects, apparatus placement may vary depending on the pattern of knee pain and tenderness. Additional apparatuses 10 may be positioned around the knee to enhance the treatment effect. In some aspects, to be most effective, the apparatus 10 may be placed over the area of pain or pathology. In other aspects, the apparatus 10 may be placed proximal or distal to the knee articulation. A similar protocol/arrangement may be applied for treatment of user's feet, ankles, legs, thighs, hips, fingers and hands, wrists, elbows, shoulders, and/or the like. In further aspects, the apparatus 59 may be combined with the apparatus 10 in additional treatment protocols. For advanced knee pathology, the apparatus 10 may be applied continuously 24 hours per day with the frequency generator 12 connected to the utility power source or enabled to be powered by a rechargeable battery within the frequency generator 12.

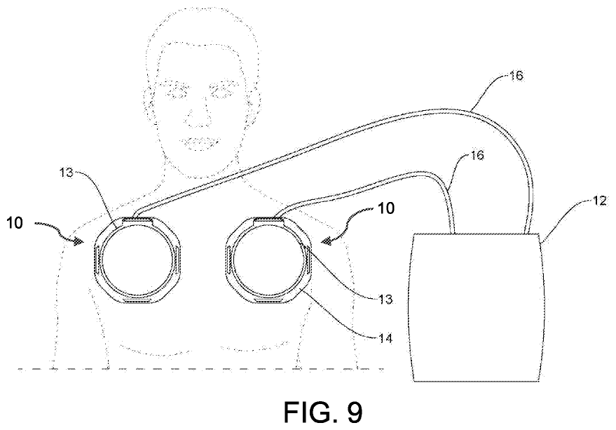

FIG. 9 depicts a second example usage of one or more light emitting apparatuses 10 in accordance with the present disclosure. In the exemplary aspect depicted in FIG. 9, the apparatuses 10 may be placed inferior to user's distal clavicle centered over acupuncture point lung one to treat pathologies of user's head and neck. The clinical effectiveness of the treatment protocol/arrangement depicted in FIG. 9 is dependent upon the specific frequencies modulating the light sources 15 in the apparatuses 10. Selecting specific frequencies that have proven effective with pulsed magnetic fields (e.g., those used in magneto therapy) have demonstrated to have greater clinical effectiveness when modulating the light sources 15. Clinical effectiveness is reflected in accelerated resolution of inflammatory markers with reduced recurrence.

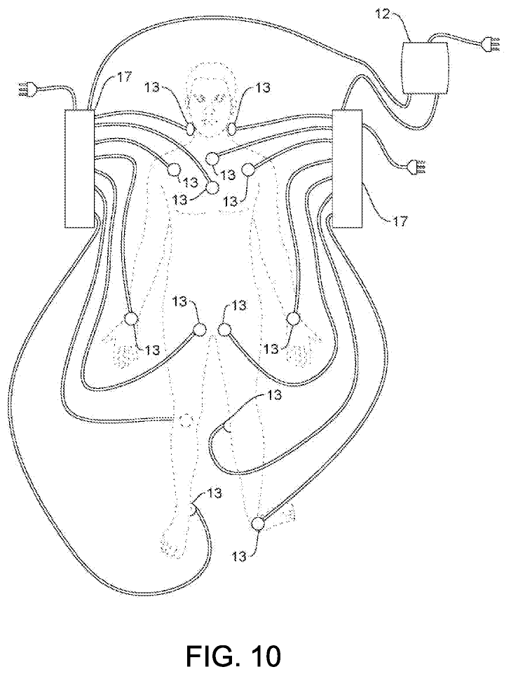

FIG. 10 depicts a third example usage of one or more light emitting apparatuses 10 in accordance with the present disclosure. Specifically, FIG. 10 depicts placement of fourteen apparatuses 10/bases 13 (including the light sources 15) bilaterally over major human peripheral arteries. In the exemplary aspect depicted in FIG. 10, the bases 13 are shown to be placed over tibial artery at the ankle, the popliteal artery at the knee, the femoral artery at the groin, the radial and ulnar arteries at the wrist, the subclavian arteries below the clavicle, the carotid arteries at the neck and over the left atrium and left ventricle of the heart. The bases 13 on each side of the user's body may be connected to the expanders 17, which in turn may be connected to the frequency generator 12. Not limited to the protocol depicted in FIG. 10, a plurality of other protocols may be developed that may place the bases 13 over acupuncture points, reflex points and referred pain points, to name a few.

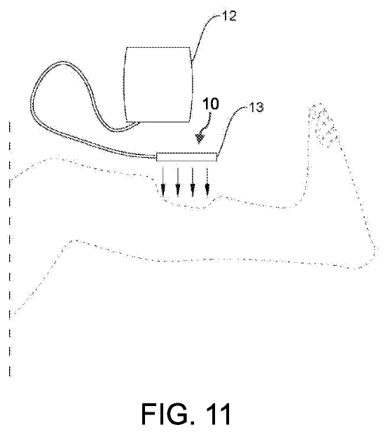

FIG. 11 depicts a fourth example usage of the light emitting apparatus 10 in accordance with the present disclosure. Specifically, FIG. 11 depicts an aspect where the apparatus 10 is placed at or applied to a human leg with an anterior leg ulceration. The wound application may vary from superficial skin tears to deep ulcerations that affect bone, muscle subcutaneous tissue and skin. The transparent surface associated with the apparatus 10 (through which the coherent light beam may be emitted by the light sources 15 to the ulceratin) may be placed directly against the wound surface or elevated off the wound surface (e.g., by 2 mm to 10 mm) by a flexible structure surrounding the apparatus 10 that may allow coherent light beam transmission directly on the wound surface. In some aspects, more than one apparatus 10 may be applied in a similar manner to ulceration depending on the wound size. As shown in FIG. 11, the apparatus 10 may be connected to the frequency generator 12 (or to the expander 17 that may be connected to the frequency generator 12) that may control the apparatus operation as described above.

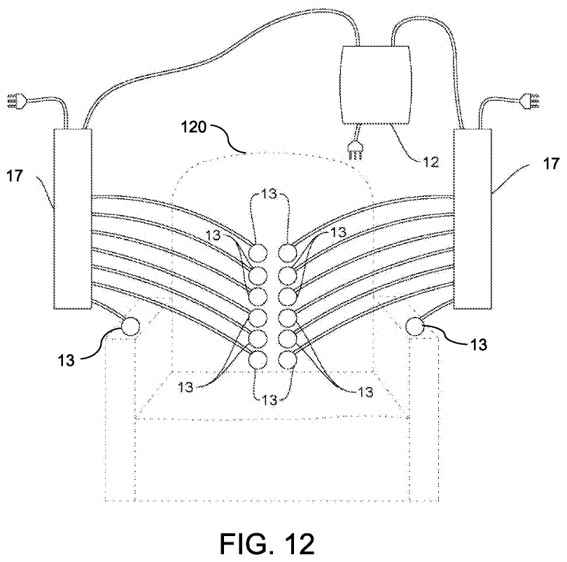

FIG. 12 depicts a fifth example usage of one or more light emitting apparatuses 10/bases 13 in accordance with the present disclosure. As shown in FIG. 12, one or more bases 13 (e.g., 12 bases 13) may be attached to a back portion of a chair 120, and two bases 13 may be attached to the chair arm rests. The bases 13 may be connected to the frequency generator 12 via the expanders 17. When the user sits on the chair 120, the apparatuses 10/bases 13 may be activated via the frequency generator 12 and made to emit coherent light modulated at the plurality of low frequencies, to treat the user. In some aspects, one or more or all of the bases 13 depicted in FIG. 12 may be replaced by the apparatuses 59 to simultaneously inject magnetic field into the user for magneto therapy.

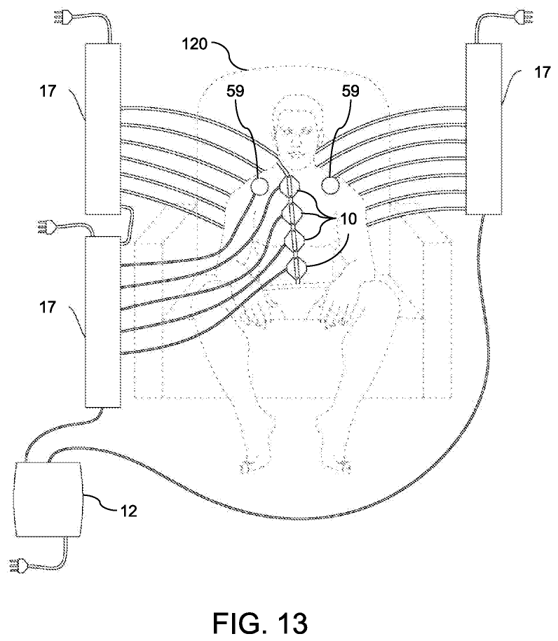

FIG. 13 depicts a sixth example usage of one or more light emitting apparatuses 10 and magnetic field generating apparatuses 59 in accordance with the present disclosure. FIG. 13 specifically depicts an aspect where the user may be sitting on the chair 120 (including one or more apparatuses 10 and/or 59 attached to the chair back portion) and two apparatuses 59 and four apparatuses 10 may be attached to anterior chest and abdomen surfaces of the user for simultaneous photo therapy and magneto therapy. The apparatuses 10 and 59 may be operated and modulated at the plurality of low frequencies by the frequency generator 12 via the expanders 17.

In some aspects, the frequency generator 12 may modulate the apparatuses 10 and 59 at those frequencies that have been proven to be clinically effective with application of electric and magnetic fields to treat user's ailment(s). The modulation of low-level laser light (i.e., the light sources 15) by means of a plurality of low frequencies has resulted in clinical outcomes that surpass the application of low frequency electromagnetic fields or low-level laser lights alone. The clinical results (described later in the description below) of synchronizing the frequencies of laser light fields and magnetic fields have demonstrated clinical benefit and effectiveness.

In some aspects, the frequency generator 12, one or more apparatuses 10 and/or one or more apparatuses 59 may collectively form a first treatment system that may enable enhanced user treatment via phototherapy and/or magneto therapy.

FIG. 14 depicts the light emitting apparatus 10 integrated with a fluid vessel 19 (or vessel 19) in accordance with the present disclosure. The vessel 19 may be attached to the apparatus 10 by using one or more straps 142 or flexible structures of predetermined material that may hold the apparatus 10 in alignment with the vessel 19. In other aspects, the vessel 19 may be attached to the apparatus 10 via one or more fasteners including, but not limited to, bolts, screws, nuts and/or the like. In an exemplary aspect, when the base 13 may be circular in shape, the vessel 19 may be shaped as a circular cylinder or a hexagonal cylinder or any shape provided the transparent top and bottom surfaces of the vessel 19 are uniformly flat and parallel, as shown in FIG. 14. In some aspects, a vessel diameter may be greater than a diameter of the base 13, so that the base 13 may be completely enclosed within the vessel's circumference periphery.

In some aspects, the apparatus 10 may be tightly integrated with the vessel 19 such that there may be no gap between an apparatus top surface (from where the coherent light emitted from the plurality of light sources 15 may exit the apparatus 10) and a vessel bottom surface. Stated another way, the apparatus 10 may be attached to the vessel bottom surface, such that there may be no gap between the surfaces of the apparatus 10 and the vessel 19 that touch each other.

A vessel top surface (or vessel first surface) and the vessel bottom surface (or vessel second surface) may be made of transparent material, so that the coherent light emitted from the plurality of light sources 15 may enter the vessel 19 through the vessel bottom surface and exit the vessel 19 from the vessel top surface. In this case, the plurality of light sources 15 may face the vessel bottom surface, such that the coherent light emitted from the plurality of light sources 15 may conveniently enter the vessel 19 via the vessel bottom surface. Based on the command signals obtained from the frequency generator 12 (not shown in FIG. 14), the light sources 15 may emit coherent light beams modulated at the plurality of low frequencies towards the vessel bottom surface. Since the vessel bottom surface is transparent, the coherent light beams may enter the vessel 19, and then exit the vessel 19 from the transparent vessel top surface.

In some aspects, the vessel 19 may be configured to store a solvent with or without a solute. In an exemplary aspect, the solvent may be water (although other polar and non-polar solvents/substances may also be used) and the solute may be one or more of, but not limited to, anticoagulated whole blood and blood plasma, biological cells and tissues, platelet rich plasma, homeopathic preparations, herbal preparations, crystals, minerals and elements, vitamins, flowers, industrial chemicals, colors, herbal and plant remedies, nutraceuticals, pharmaceuticals, biomolecular substances, flower essences, essential oils, gem stones, and/or the like. The coherent light beams entering the vessel 19 from the vessel bottom surface may interact with the solvent with or without the solute before exiting from the vessel top surface. In some aspects, any substance may be the solute as long as the coherent light beams (i.e., the laser light) are not completely blocked by the solute.

During operation, the apparatus 10 (including the light sources 15) may be integrated or attached with the vessel 19 in the arrangement described above. The light sources 15 may then emit coherent light beams into the vessel 19 (storing the solvent with or without the solute) via the vessel bottom surface based on command signals obtained from the frequency generator 12. Since the vessel 19 may be storing the solvent with or without the solute, the coherent light beams may interact with the solvent and/or the solute and may then exit the vessel 19 via the vessel top surface. The vessel top surface may be disposed in proximity to a human body or an animal body, and the coherent light beams escaping the vessel top surface may penetrate into the animal skin or human skin for bio-stimulating phototherapy. In this manner, the coherent light beams entering the animal skin or human skin may be modulated by the solvent (with or without the solute) that may be stored in the vessel 19.

A person ordinarily skilled in the art may appreciate that laser light beams (e.g., the coherent light beams emitted by the light sources 15) may be modulated by two methods, a direct modulation method and an indirect modulation method. The direct modulation method utilizes the frequency generator 12 using which the laser light beams may be modulated at their source (i.e., at the light sources 15) by controlling the amount of current passed to the light sources 15. The description described above in conjunction with FIGS. 1-13 relates to the direct modulation method of the laser light beams or coherent light beams emitted from the light sources 15.

In the indirect modulation method (performed in the present disclosure independently over the direct modulation method described above), the coherent light beam or the laser light beam gets modulated after the laser light beam has been emitted by the light source 15. In accordance with the present disclosure, the laser light beams emitted from the light sources 15 get modulated by the indirect modulation method when the laser light beams interact with the solvent and/or the solute present in the vessel 19. In some aspects, the laser light modulation by the solvent and/or the solute present in the vessel 19 is different from two conventional modulation processes known to a person ordinarily skilled in the art, i.e., absorptive modulation and refractive modulation. The laser light modulation by the solvent and/or the solute present in the vessel 19 is a result of trifold interaction of the laser light beam emitted by the apparatus 10, the solvent, and the solute present in the vessel 19, which leads to enhanced laser light phototherapy.

Specifically, a person ordinarily skilled in the art may appreciate that liquid water (i.e., the solvent included in the vessel 19) has two distinct phases, i.e., bulk liquid water and crystalline liquid water. Each distinct phase of water forms domains that separate the two forms of water. The physical properties of liquid crystalline water domains are different from the physical properties of bulk water domains. The liquid crystalline water has been reported to have a higher index of refraction, a higher T2 relaxation time in MRI scans (time required for the transverse magnetization to fall to approximately 37% of its initial value) and a higher electrical potential. In addition, the liquid crystalline water has greater viscosity with increased molecular alignment than the bulk liquid water.

The infrared laser light is known to interact with and expand the domains of the liquid crystalline water. Withdrawing the radiant energy reverses the liquid crystalline domain expansion. Radiant energy is stored in the liquid crystalline water domains as entropy loss and charge separation. The absorbed energy from infrared laser radiation builds liquid crystalline zones around charged particles and thereby is a force that separates charges. The separated charges generate forces that drive particle movements. The liquid crystalline domains surrounding the charged molecules, membranes and biological structures oscillate in resonance with the electromagnetic information specific to the molecules, the membranes and the biological structures. The interaction of the oscillating electromagneic fields within the liquid crystalline domains modulates the laser radiation that may be radiating through the liquid crystalline domain. The oscillating electromagnetic fields may be influenced by the dynamic interaction between the frequency specific laser radiation (e.g., the laser light beams emitted from the light sources 15) and the solvent, between the frequency specific laser radiation and the solute and between the molecular interactions of the solvent and the solute. The specifics of the solvent and the solute determine the characteristics of the oscillating electromagnetic field within the liquid crystalline water domains. These dynamics of the trifold interaction described above results in a complex phase shift of the plurality of laser beams generated by the light sources 15 radiating through the vessel 19. This form of indirect optical modulation is different from the absorptive modulation and refractive modulation that is known to those skilled in the art of laser modulation.

In further aspects, a transparent decal with a black ink (laser absorbing) geometric pattern (not shown) may be added/attached to the vessel top surface to cause additional indirect optical modulation of the coherent light beams (i.e., laser lights) exiting the vessel top surface. In an exemplary aspect, the geometric pattern may be spiral with a preset count of turns with different line densities. When the coherent light beams escaping the vessel top surface (after interacting with the solvent and/or the solute stored in the vessel 19) pass through the geometric pattern and then enter the human/animal skin, the light beams generate enhanced phototherapy response. A person ordinarily skilled in the art may appreciate the principle described here with the classical laser constructive and destructive patterns that lasers exhibit (wave nature of light) when an object is in the laser's path.

In some aspects, the vessel 19 may further include one or more ports (not shown) that may be sealed with fluid tight caps to allow fluids (e.g., the solvent with or without the solute) to be poured into and removed from the vessel 19. The exemplary vessel shape depicted in FIG. 14 should not be construed as limiting. The vessel 19 may have any other shape without departing from the present disclosure scope. The surface (i.e., the vessel bottom surface) that receives the incoming laser radiation should be parallel to the surface (i.e., the vessel top surface) through which the laser radiation exits from the vessel 19.

In some aspects, the frequency generator 12, the apparatus 10 and the vessel 19 may collectively form a second treatment system that may enable enhanced user treatment via phototherapy.

FIG. 15 depicts two light emitting apparatuses 10 (with or without the vessel 19) connected with an intravenous solution bag 152 (or intravenous solution vessel) in accordance with the present disclosure. The intravenous solution bag 152 (or bag 152) may be configured to store and administer intravenous fluids to patients. The bag 152 may be made of transparent material that may allow coherent light beams (e.g., the laser light beams emitted from the light sources 15) to enter the bag 152. A first surface (e.g., a left sidewall surface) associated with the bag 152 may be attached with one or a first apparatus 10 and a second surface (e.g., a right sidewall surface) associated with the bag 152 may be attached with another or a second apparatus 10. Stated another way, the first apparatus 10 may be disposed in proximity to the first surface and the second apparatus 10 may be disposed in proximity to the second surface associated with the bag 152. The bag 152 may receive the coherent light beams from the first and second apparatuses 10 via respective first and second surfaces. In the exemplary aspect depicted in FIG. 15, the first and second surfaces are opposite to each other on the bag 152. In some aspects, a plurality of apparatuses 10 may be attached to the bag 152 by application of the extender/expander 17 based upon the surface area of the bag 152.

The two or more apparatuses 10 attached to the bag 152 may operate based on the command signals obtained from the frequency generator 12, as described above. Specifically, based on the command signals obtained from the frequency generator 12, the two or more apparatuses 10 may emit pulses of coherent light beams into a bag interior portion via the transparent first and second surfaces. The bag interior portion may store intravenous fluids or the solvent (with or without the solute) that may receive the pulses of coherent light beams and undergo chemical/molecular reaction described above.

In some aspects, the bag 152 may be attached with or connected to an intravenous tube 154 and an intravenous catheter (not shown) that may inserted into a user's peripheral vein. The intravenous tube 154 and the intravenous catheter may be configured to inject or transfer the intravenous fluids or the solvent (with or without the solute), which may have interacted with the pulses of coherent light beams emitted from the two or more apparatuses 10, into the user's peripheral vein (or human/animal body) for user's treatment.

In some aspects, the frequency generator 12, the two or more apparatuses 10, the bag 152, the intravenous tube 154 and the intravenous catheter may collectively form a third treatment system that may enable enhanced user treatment via intravenous fluid injection.

Examples of a few clinical experiments using the apparatus 10 and/or the apparatus 59 and/or the vessels/bag 19/152 are described below. The examples are for illustrative purpose only and should not be construed as limiting or exhaustive.

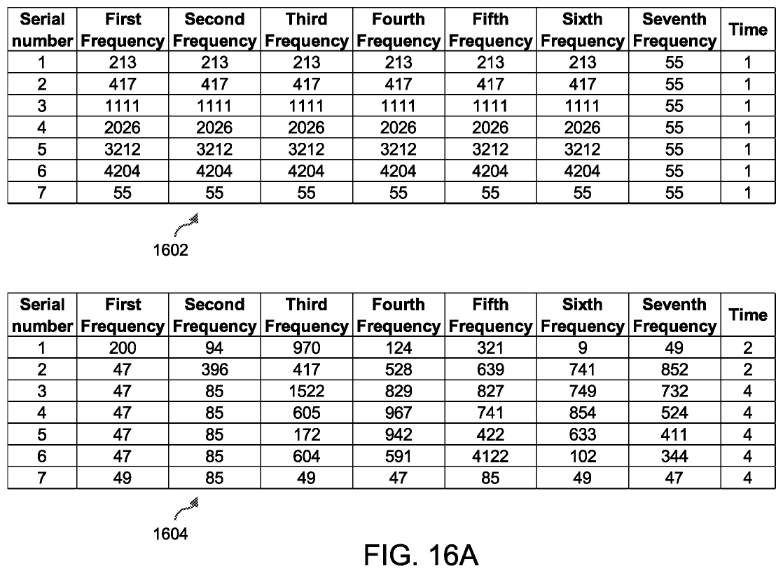

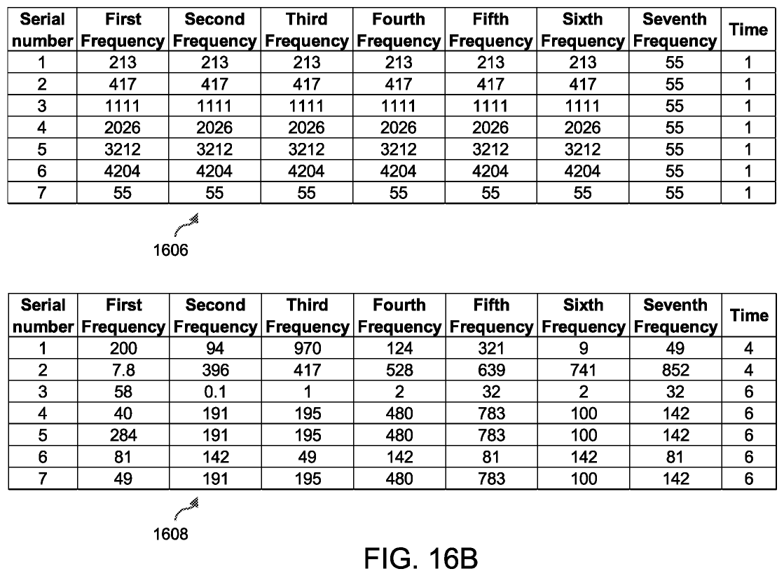

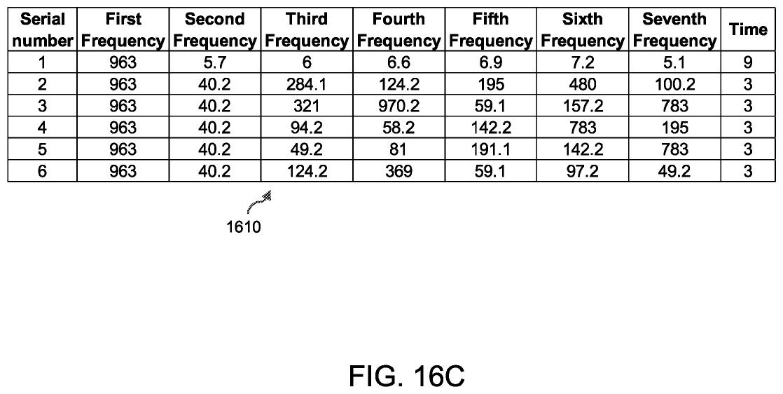

In a first clinical experiment, phototherapy application of 650 nm laser light emitted from the apparatus 10 radiated through the vessel 19 was performed on a 73-year-old male with a 14 year history of Parkinson's Disease. Medication to control the symptoms of Parkinson's disease included Amantadine HCL 100 mg once per day, Carbidopa-Levodopa CR 50-200 mg five times per day and Carbidopa-Levadopa 25-100 mg six times per day. Ten years ago, surgical implantation of a deep brain stimulator was required to provide additional control of the symptoms of Parkinson's disease. Frequency modulated laser light discs (or the apparatuses 10) were initiated with placement of one laser discs inferior to the right distal clavicle and the second laser disc over the left distal clavicle for 4 hours in the AM and 4 hours in the PM. The frequencies and time duration of two frequency files are presented in tables 1602 and 1604 depicted in FIG. 16A. The laser discs were radiated though fluid vessels (e.g., the vessels 19 or 152) filled with water solvent and one 50-200 mg Carbidopa-Levadopa CR tablet and one 25-100 Carbidopa-Levadopa tablet was dissolved in the aqueous solution. After three weeks, the patient reported an improvement in his gait with increased stability and balance. The patient noted a reduction in his musculoskeletal rigidity and shaking symptoms. The patient continued taking the photobiology therapy.

In a second clinical experiment, a 76-year-old male with a 6 year history of progressive knee pain was treated with the apparatuses 10 and 59 and the vessel 19. The patient had negative history of prior surgical intervention, and had two past intra articular cortisone injections. The first injection provided several months of pain relief. The second cortisone injection provided several weeks of pain relief. The use of topical over-the-counter anti-inflammatory creams provided temporary pain relief. The patient was asked to consider the possibility of total knee replacement if the symptoms prevented him from routine activities of daily living. Frequency modulated laser light discs (e.g., the apparatuses 10) were initiated with placement of the laser discs on the medial and lateral sides of his knee while he slept. The frequencies and time duration of the frequency files are depicted in tables 1606 and 1608 in FIG. 16B. In addition, a portable pulsed frequency specific magnetic field generator (e.g., the apparatus 59) was applied during the daytime with the same frequency programs. After applying the frequency specific laser discs at night and the pulsed magnetic field generator during the day for several weeks with modest pain relief, fluid vessels (i.e., the vessel 19) with water solvent and one 5 mg Prednisone tablet dissolved in the aqueous solution were added to the laser discs. After application for a week using the protocol described above, the patient reported reduced pain. The patient continued taking the photobiology therapy with progressive reduction in pain and improved range of motion.

In a third clinical experiment, an 83-year-old female presented with a five to six year history of progressive right knee pain localized to the medial and lateral joint line. Radiographs showed three compartment degenerative osteoarthritic changes. On examination, the knee was without effusion but painful to palpation localized to the anteromedial and anterolateral joint line. Knee was stable with reference to the ACL and the PCL ligaments but opened 3 to 5 degrees with valgus stress applied. Weight bearing was painful requiring the use of a cane. Frequency modulated low level laser light disc (i.e., the apparatus 10) was initiated with placement of two laser discs centered over the medial and the lateral joint line. The frequencies and time duration of frequency files is shown in table 1610 of FIG. 16C. Within 30 minutes of applying coherent laser light using the apparatus 10, the pain was reduced significantly upon weight bearing to tolerance. Patient was sent home with two laser discs and a frequency generator with instructions to apply the low-level laser light discs to the medial and lateral aspect of her knee while she slept. The patient applied the low-level laser light therapy nightly for the next two weeks with resolution of the knee inflammation. With the understanding of the structural changes of the knee articulation reflected in the radiographic and clinical findings, the patient was encouraged to continue the application of the low-level laser several hours during the day. At the three month follow up, the patient reported that the pain was completely resolved. Patient continued to apply the two laser discs several times per week for several hours at home.

In the above disclosure, reference has been made to the accompanying drawings, which form a part hereof, which illustrate specific implementations in which the present disclosure may be practiced. It is understood that other implementations may be utilized, and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a feature, structure, or characteristic is described in connection with an embodiment, one skilled in the art will recognize such feature, structure, or characteristic in connection with other embodiments whether or not explicitly described.

Further, where appropriate, the functions described herein can be performed in one or more of hardware, software, firmware, digital components, or analog components. For example, one or more application specific integrated circuits (ASICs) can be programmed to carry out one or more of the systems and procedures described herein. Certain terms are used throughout the description and claims refer to particular system components. As one skilled in the art will appreciate, components may be referred to by different names. This document does not intend to distinguish between components that differ in name, but not function.

It should also be understood that the word “example” as used herein is intended to be non-exclusionary and non-limiting in nature. More particularly, the word “example” as used herein indicates one among several examples, and it should be understood that no undue emphasis or preference is being directed to the particular example being described.

A computer-readable medium (also referred to as a processor-readable medium) includes any non-transitory (e.g., tangible) medium that participates in providing data (e.g., instructions) that may be read by a computer (e.g., by a processor of a computer). Such a medium may take many forms, including, but not limited to, non-volatile media and volatile media. Computing devices may include computer-executable instructions, where the instructions may be executable by one or more computing devices such as those listed above and stored on a computer-readable medium.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating various embodiments and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their ordinary meanings as understood by those knowledgeable in the technologies described herein unless an explicit indication to the contrary is made herein. In particular, use of the singular articles such as “a,” “the,” “said,” etc. should be read to recite one or more of the indicated elements unless a claim recites an explicit limitation to the contrary. Conditional language, such as, among others, “can,” “could,” “might,” or “may,” unless specifically stated otherwise, or otherwise understood within the context as used, is generally intended to convey that certain embodiments could include, while other embodiments may not include, certain features, elements, and/or steps. Thus, such conditional language is not generally intended to imply that features, elements, and/or steps are in any way required for one or more embodiments.

That which is claimed is:

1. A light emitting apparatus comprising:
a base; and
a plurality of light sources disposed on the base, wherein:
each light source is configured to emit coherent light,
the plurality of light sources comprises a first set of light sources and a second set of light sources,
the first set of light sources is configured to emit pulses of coherent light modulated at a first frequency from a plurality of frequencies, based on command signals obtained from a frequency generator,
the second set of light sources is configured to emit pulses of coherent light modulated at a second frequency, from the plurality of frequencies, based on the command signals obtained from the frequency generator, and
the first frequency is different from the second frequency.

2. The light emitting apparatus of claim 1, wherein each light source from the plurality of light sources is a laser light source.

3. The light emitting apparatus of claim 1, wherein the light emitting apparatus is configured to be attached to an animal body or a human body for bio-stimulating phototherapy, and wherein the coherent light emitted by each light source is configured to penetrate animal skin or human skin.

4. The light emitting apparatus of claim 1, wherein the plurality of frequencies is in a range of 0.1 Hz to 5,000 Hz.

5. The light emitting apparatus of claim 1, wherein each light source is configured to emit the coherent light with a wavelength of 390 to 1,600 nm.

6. The light emitting apparatus of claim 1, wherein the plurality of light sources further comprises:
a third set of light sources configured to emit pulses of coherent light modulated at a third frequency from the plurality of frequencies, based on the command signals obtained from the frequency generator;
a fourth set of light sources configured to emit pulses of coherent light modulated at a fourth frequency from the plurality of frequencies, based on the command signals obtained from the frequency generator;
a fifth set of light sources configured to emit pulses of coherent light modulated at a fifth frequency from the plurality of frequencies, based on the command signals obtained from the frequency generator;
a sixth set of light sources configured to emit pulses of coherent light modulated at a sixth frequency from the plurality of frequencies, based on the command signals obtained from the frequency generator; and
a seventh set of light sources configured to emit pulses of coherent light modulated at a seventh frequency from the plurality of frequencies, based on the command signals obtained from the frequency generator,
wherein the first frequency, the second frequency, the third frequency, the fourth frequency, the fifth frequency, the sixth frequency and the seventh frequency are different from each other.

7. The light emitting apparatus of claim 6, wherein each of the first set of light sources, the second set of light sources, the third set of light sources, the fourth set of light sources, the fifth set of light sources, the sixth set of light sources and the seventh set of light sources comprises six light sources.

8. The light emitting apparatus of claim 6, wherein each of the first set of light sources, the second set of light sources, the third set of light sources, the fourth set of light sources, the fifth set of light sources, the sixth set of light sources and the seventh set of light sources is disposed in a hexagonal pattern on the base.

9. The light emitting apparatus of claim 6, wherein each of the first frequency, the second frequency, the third frequency, the fourth frequency, the fifth frequency, the sixth frequency and the seventh frequency is further modulated at an eighth frequency.

10. The light emitting apparatus of claim 9, wherein the eighth frequency is less than 16 Hz.

11. The light emitting apparatus of claim 9, wherein the eighth frequency is less than lowest of the first frequency, the second frequency, the third frequency, the fourth frequency, the fifth frequency, the sixth frequency and the seventh frequency.

12. The light emitting apparatus of claim 1, wherein the base is shaped as a circular disc.

13. The light emitting apparatus of claim 1, wherein each light source has an associated power rating of less than 5 milliwatt (mW).

14. A system comprising:
a frequency generator; and
a light emitting apparatus comprising:
a base; and
a plurality of light sources disposed on the base, wherein:
each light source is configured to emit coherent light,
the plurality of light sources comprises a first set of light sources and a second set of light sources,
the first set of light sources is configured to emit pulses of coherent light modulated at a first frequency from a plurality of frequencies, based on command signals obtained from the frequency generator, and
the second set of light sources is configured to emit pulses of coherent light modulated at a second frequency, from the plurality of frequencies, based on the command signals obtained from the frequency generator, wherein the first frequency is different from the second frequency.

15. The system of claim 14, wherein the light emitting apparatus is configured to be attached to an animal body or a human body for bio-stimulating phototherapy, and wherein the coherent light emitted by each light source is configured to penetrate animal skin or human skin.

16. The system of claim 14 further comprising a magnetic field generating apparatus comprising:
a first magnetic coil configured to generate pulses of magnetic field at the first frequency based on the command signals obtained from the frequency generator, wherein the pulses of magnetic field generated by the first magnetic coil are synchronized with the pulses of coherent light emitted by the first set of light sources; and
a second magnetic coil configured to generate pulses of magnetic field at the second frequency based on the command signals obtained from the frequency generator, wherein the pulses of magnetic field generated by the second magnetic coil are synchronized with the pulses of coherent light emitted by the second set of light sources.

17. A system comprising:
a frequency generator;
a light emitting apparatus comprising:
a base; and
a plurality of light sources disposed on the base, wherein:
each light source is configured to emit coherent light,
the plurality of light sources comprises a first set of light sources and a second set of light sources,
the first set of light sources is configured to emit pulses of coherent light modulated at a first frequency from a plurality of frequencies, based on command signals obtained from the frequency generator, and
the second set of light sources is configured to emit pulses of coherent light modulated at a second frequency, from the plurality of frequencies, based on the command signals obtained from the frequency generator, wherein the first frequency is different from the second frequency; and
a magnetic field generating apparatus comprising:
a first magnetic coil configured to generate pulses of magnetic field at the first frequency based on the command signals obtained from the frequency generator, wherein the pulses of magnetic field generated by the first magnetic coil are synchronized with the pulses of coherent light emitted by the first set of light sources; and
a second magnetic coil configured to generate pulses of magnetic field at the second frequency based on the command signals obtained from the frequency generator, wherein the pulses of magnetic field generated by the second magnetic coil are synchronized with the pulses of coherent light emitted by the second set of light sources.

18. The system of claim 17, wherein the light emitting apparatus is configured to be attached to an animal body or a human body for bio-stimulating phototherapy, and wherein the coherent light emitted by each light source is configured to penetrate animal skin or human skin.

19. The system of claim 17, wherein the magnetic field generating apparatus is configured to be attached to an animal body or a human body, and wherein the pulses of magnetic field generated by the first magnetic coil and the second magnetic coil are configured to penetrate animal skin or human skin.

20. The system of claim 17, wherein the plurality of frequencies is in a range of 0.1 Hz to 5,000 Hz.
